(19) United States
(12) Patent Application Publication
(10) Pub. No.: US 2008/0177169 A1
(43) Pub. Date: Jul. 24, 2008

(54) ANGULAR FILTERS FOR OPTICAL TOMOGRAPHY OF HIGHLY SCATTERING MEDIA

(76) Inventors: Glenn Harrison Chapman, Coquitlam (CA); Bozena Kaminska, Vancouver (CA); Paulman Konn Yan Chan, Coquitlam (CA); Fartash Vasefi, Burnaby (CA); Nicholas Pfeiffer, Mission (CA)

Correspondence Address: FASKEN MARTINEAU DUMOULIN, LLP, 2900 - 550 Burrard Street, VANCOUVER, BC, V6C 0A3, omitted

(21) Appl. No.: 11/625,244

(22) Filed: Jan. 19, 2007

(51) Int. Cl. A61B 5/05

(52) U.S. Cl. 600/407

(57) ABSTRACT

An angular filter is provided, including a straight tunnel within a material, the tunnel having a roughened surface. When scattered light enters the tunnel, and strikes the roughened surface at a shallow angle, the scatter light is further scattered within the tunnel, striking the surface of the tunnel a multiplicity of times. Each such strike partially absorbs the light ray, such that the intensity of the scattered light exiting the tunnel is minimized.

### FIELD OF THE INVENTION

This invention relates to the creation and use of angular filters, and more particularly to angular filters that restrict light reaching sensors to very small acceptance angles.

### BACKGROUND OF THE INVENTION

It has long been a goal to have optical detection techniques that supplement or replace X-rays for imaging objects within tissue. Medical optical tomography techniques depend on the fact that light penetrates quite deeply into tissue, where a small amount of light is absorbed, but most of the light becomes heavily scattered. The light is separated into the following categories:
a) highly scattered light, which is light, scattered by the medium whose path is randomly deviated from its original path making it difficult to extract the internal structural of the medium.
b) unscattered or ballistic light, which is light that is not scattered by the medium. This carries information about the structure of the tissue through which it passes. However typically it is millions of time weaker than the scattered light.
c) slightly scattered light, also called quasi-ballistic or snake photons which is light scattered by the medium, but whose path zig zags or snakes around the unscattered light path but deviates only slightly from it. Slightly scattered light arises because in many media light has a statistically higher probability of scattering towards the forward direction. Quasi-ballistic light also carries structural information and will typically have a much higher intensity than ballistic photons, but again at many orders of magnitude less than the scattered light.

For detection of objects or structures located within a scattering medium the ballistic or quasi ballistic light will be affected by the object, such as when the light is absorbed or blocked by a solid object. Hence it is possible to image an object by detecting this ballistic or quasi-ballistic light. However, scattered light levels emerging from the medium are almost unaffected by the presence of the object, and hence contain almost no information about the internal structure.

Optical imaging techniques have using light have several important advantages over X-rays for non-invasive imaging of interior body structures:

1) Light is non-ionizing at wavelengths in the visible to near-infrared range (˜500-1200 nm). When the optical intensity is kept below well-known safety limits for thermal damage, light does not affect tissue during short exposures. Thus, optical techniques could allow for greater monitoring frequency, enhancing early detection of cancers.
2) The absorbed or scattering of light by tissue varies with wavelength, type of material, and biological state. Using light thus allows for technical options that X-ray does not, so it is possible to design a system that can direct several wavelengths through the tissue into a single detection system at the output. Thus optical characteristics of tissue can be measured at varying wavelengths, providing important biomedical and functional information.
3) Optical imaging techniques are compatible with Computer-Aided Tomography (CAT). When unscattered data is detected with sufficient sensitivity, mathematical analysis can generate three dimensional tissue images, as with CAT scanners. Digital optical detectors cover two dimensional areas more rapidly than do X-ray CAT systems, giving advantages such as quick image storage, transport and analysis.
4) Laser diode light sources of many wavelengths are available, are compact, and require little power, creating the potential for new portable medical and industrial imaging systems.

While optical techniques offer many potential improvements over the existing X-ray and acoustic methods, prior art optical systems have problems that have prevented commercial application to date. Imaging the contents and, or structure within a scattering medium, such as tissue, can be an extremely challenging problem. Scattering in a medium is exponentially related to the depth of the medium and thus, scattering levels can reach extremely high levels (many orders of magnitude more scattered photons than non-scattered photons) for many applications such as medical imaging of human tissues. In a typical example, when imaging through 5 cm of breast tissue, the scattered light is about one hundred billion times greater than the unscattered or slightly scattered light, which contains the structural information. This extremely high scattering can completely obscure the contents of the medium from view, because the scattered photons can be considered noise photons for imaging purposes.

When trying to observe the contents of a scattering medium, several methods have been employed. The most common methods used are Time-Domain and Coherence-Domain Tomography. Both of these methods use only those photons that travel through the medium with the shortest path, because highly scattered light inherently must travel a longer path.

This has suggested to practitioners of the art the use of time of travel, or Time Domain Tomography, which measures the transmitted light generated by femtosecond laser pulses into a scattering medium and looks for the earliest arriving photons. The ballistic/quasi-ballistic photons arrive first, having traveled the shortest distance, while scattered photons arrive hundreds of picoseconds later. U.S. Pat. No. 5,275,168 to Reintjes, et al. describes an optical setup to create the time gating methodology for use with Raman spectroscopy. U.S. Pat. No. 6,795,199 to Suhami describes the use of Time Domain Tomography to obtain both structural and depth information in the tissue of the human eye. The problem with this technology is that light has a very high velocity and thus the times required to discriminate path differences of only millimeters is about 300 femtoseconds. This requires expensive ultra short pulse duration lasers as the light sources and expensive femtosecond optical shutters.

One alternative used to avoid such high speed systems is to employ the concept that with a coherent optical source, differences in path lengths also create differences in the phase of the light. This led to Optical Coherence Tomography (OCT), which employs the interference created by the difference in phase between a reference beam and a beam of light that enters a scattering medium and that is reflected back along the same direction. For example, U.S. Pat. No. 5,491,524 to Hellmuth, et al. describes the use of a rotating mirror and lens system that optically scans cornea tissues with a light source and combines the returned light with the reference beam, creating maps of structure within the tissue at specific depths. While OCT is very good at giving fine detailed structure information about relatively shallow tissue depths (few millimeters), it has difficulty imaging very thick structures. Also, longer distances require the use of long coherence length laser sources and careful control of the reference beam path.

Another technique is Diffuse Optical Tomography, which treats the imaging of a scattering medium as an inverse problem, and uses multiple measurements of the medium with sources and detectors at various positions to develop and parameterize a three-dimensional model that describes the contents of the medium. U.S. Pat. No. 5,137,355 to Barbour et al. entitled “Method of Imaging a Random Medium” describes the use of multi-wavelength images taken of scattered light from a tissue and an analysis algorithm to calculate the blood oxygenation level in a given volumes U.S. Pat. No. 4,810,875 to Wyatt entitled “Method and apparatus for examining the interior of semi-opaque objects” describes the illumination of a scattering medium with a laser beam, and collection of the resulting surface light from many locations, which is then analyzed by an algorithm to estimate the location of objects. U.S. Pat. No. 4,555,179 to Langerhole et al., entitled “Detection and imaging of objects in scattering media by light irradiation” describes the illumination of a scattering medium with a collimated beam and detection of changes in the back reflection to calculate the location and depth of objects in a scattering medium.

An alternative method is to make use of the directional or angular distribution of the light leaving the scattering medium. Angular filters are designed to extract all photons that arrive at a detector within very small acceptance angles. The simplest form of an angular filter is a collimator consisting of a set of aligned circular apertures in two light blocking baffles. These aligned holes create a narrow angle of acceptance of incoming light given by the angle formed between the aperture diameter d, and the collimator length L by the formulas:

Aspect ratio=d/L

Acceptance angle=arc tan(d/L)

Thus, an angular filter, with two 50 μm diameter aligned holes spaced by 10 mm length, has an aspect ratio of 1:200 and an acceptance angle of 0.29°. If a photon emerges from a scattering medium with an angle that is greater than the acceptance angle, then ideally it either gets absorbed within the angular filter or fails to enter it. One common system arrangement is to align a collimated light source, such as that emerging from a laser, with the angular filter holes and place a scattering medium between the laser source and the angular filter. With this arrangement, as a first approximation, the unscattered light would not be attenuated by the angular filter. However highly scattered light emerging from the medium tends to be uniformly distributed. The smaller the acceptance angle, the greater the resultant rejection of scattered light, for in an angular filter, the fraction of the scattered light that will pass through the angular filter will vary directly with the square of the acceptance angle. Thus the smaller the acceptance angle, the less scattered light passes through, while the ballistic photons are unaffected by the acceptance angle. Hence, when the acceptance angle is very small, for example an angle of less than one degree, the rejection of the scattered light becomes very high. Such an illumination method is referred to as “trans-illumination”.

Collimators have a long history of investigation. U.S. Pat. No. 6,987,836 to Tang et al., entitled “Anti-scatter grids and collimator designs, and their motion, fabrication, and assembly” describes the use of collimators for the enhancement of imaging with electromagnetic radiation. Tang describes a grid of solid integrated walls that act as collimators for detecting electromagnetic radiation which is intended for use with x-ray and gamma-ray imaging. U.S. Pat. No. 6,420,709 to Block et al., entitled “Methods of minimizing scattering and improving tissue sampling in non-invasive testing and imaging,” describes a procedure and apparatus for optically testing the concentration in samples. Collimating optics, along with two openings or apertures placed after the scattering medium being optically examined, are used to eliminate a high proportion of scattered light while allowing ballistic and snake-like radiation to be transmitted. U.S. Pat. No. 5,231,654 to Kwasnick et al., entitled “Radiation imager collimator” describes a collimator for use in an imaging system based on a radiation point source, which consists of a collection of tunnels orientated along angles that correspond to the direct beam paths from laser source to the radiation detector. These collimators are formed out of a photosensitive material that is coated with a radiation absorbent material. Another approach, described in U.S. Pat. No. 4,288,697 to Albert, entitled “Laminate radiation collimator”, involves the use of layers of openings with particular spacings between them, with one layer stacked on top of another multiple times, so as to only transmit x-rays along particular paths as determined by the positioning of the layered openings.

Collimators can also be used to improve spatial resolution when imaging the contents of a medium by imposing constraints, with respect to the detector, on the angle of incidence and/or the region of origin of the photons. U.S. Pat. No. 6,940,070 to Turner, entitled “Imaging probe”, describes a honeycomb collimator in conjunction with a gamma camera detector to locate sentinel lymph nodes. The collimator is interchangeable for either higher sensitivity or higher spatial resolution, and is to be used with direct contact to the tissue, or even inside a surgical incision. U.S. Pat. No. 6,353,227 to Boxen, entitled “Dynamic collimators”, describes the use of an aperture-based collimator to spatially restrict the passage of any photon or material particle to achieve a desired detection resolution. The collimator has a plurality of apertures of pre-selected cross-sectional shape and three-dimensional distribution so as to restrict the passage of photons or particles according to defined collimated paths.

U.S. Pat. No. 4,125,776 to Tosswill et al., entitled “Collimator for X and gamma radiation” describes a collimator consisting of a glass mosaic substrate comprised of a plurality of glass columns aligned in parallel, forming passages between the columns with at least a 5-to-1 length-to-width aspect ratio. The walls of each column are to be coated with a metal having an absorption coefficient of at least 14, and each of the glass columns is to have within it a radiation absorbing chemical compound with an absorption coefficient such that the product of the absorption coefficient and the column length in centimeters is at least 12. With these parameters, the fraction of radiation that passes through the column walls together with the fraction of radiation that passes through the collimator by traveling entirely within the glass columns is not more than 1% of the fraction of radiation that passes through the collimator entirely within the passages.

While a pair of aligned collimator holes works as a single detector, arrays of aligned holes in two baffles will not work as an angular filter because a fraction of the scattered light from any of the first baffle holes will pass through all the holes in the second baffle. To compensate for this an angular filter array may use high-aspect ratio tunnels or tunnels of diameter d spaced at width w through a solid material of length L to create pathways through which photons can pass unattenuated if they arrive within an allowable acceptance angle with respect to the longitudinal axis of each tunnel. Photons that arrive with incident angles beyond the acceptance angle will strike the tunnel sides and ideally be attenuated. The detector for an array angular filter will be a linear or two dimensional array imager such as a CCD) or CMOS imaging pixel array. Each pixel of this array must be the same size or smaller than the spacing w of the tunnels. Pixels smaller than the spacing w will increase the resolution of system, but ideally the number of pixels in a given tunnel should be an integer multiple of spacing w. To create a two dimensional image a linear array and possibly the source, will need to be scanned in a direction perpendicular to the array width. Practitioners of the art will recognize that with an array angular filter the light source may be a collimated regular light source, a single laser, multiple lasers, or a single laser beam expanded to cover the full width of the array. Ideally optical methods well known to practitioners of the art should be used to restrict the light as nearly as possible to the area or the array. Light outside of that area adds to the scattered background but does not contribute ballistic or quasiballistic photons. Also such practitioners will recognize that for any given aspect ratio in an angular filter the minimum size of objects that can be resolved will be set by the diameter of the tunnels, the size of the pixels in the detector, and the scattering level in medium.

U.S. Pat. No. 5,726,443 to Immega, et al., describes a method of increasing the aspect ratio of an angular filter of high aspect ratio tunnels, through the addition of baffles placed at specific spacings provided by a formula that depends on the filter parameters. Also noted is that such arrays may include tunnels set at different angles.

It is known that the detection limit in scattering media for any angular filter is set by the rate at which it removes the scattered light while passing the unscattered and slightly scattered light. There is always some scattered light that has the correct angles to pass through the angular filtration or in a collimator. This scattered light appears as a uniform noisy background which obscures the structural information carried by the unscattered light. The key is to remove as much of that scattered light as possible.

It is also known that an excellent method of producing such high-aspect-ratio small hole collimating structures is by using micromachining. Micromachining is a technology that uses integrated circuit fabrication methods to create mechanical and optical structures of micron dimensions. In one example in the literature, a large length of the array 51 μm lines on 102 μm spaces were created for a 10 mm, creating a 200:1 aspect ratio and 0.29° acceptance angle. This combination of long length and small hole size suggested that silicon surface micromachining could best generate the structure. A silicon wafer was patterned with the 51 μm lines on 102 μm spaces in a 20×10 mm area. These were created using isotropic surface etching of the silicon. The array was assembled from two 0.5 thick wafers of silicon creating the high aspect ratio angular filter. Practitioners of the art will recognize there are many other methods of creating such stub 100 micron diameter tunnels.

### SUMMARY OF THE INVENTION

The optical angular filters according to the invention are made from high-aspect ratio tunnels or tunnels through a solid material to create pathways through which photons can pass unattenuated if the photons arrive within an allowable acceptance angle with respect to the longitudinal axis. These may be single tunnels, a linear array of tunnels, or a two dimensional array of such tunnels. The greater the aspect ratio, the more scattered light rejected in the angular filter. In addition, for a given aspect ratio the minimum size of objects that can be resolved will be determined by the diameter of the tunnels. The angular filters according to the invention are particularly useful, but not limited to, cases wherein the diameter of the tunnels is in the range of hundreds of microns or less, and aspect ratios are greater than 50. However angular filters according to the invention can be used with different aspect ratios and diameters.

As highly scattered light is nearly homogenously distributed in all directions there is a significant amount of illumination that enters the angular filter that is at small, shallow, angles slightly larger than the allowed acceptance angle. If that shallow angle scattered light is not absorbed by the angular filter wall, hut instead is reflected, it can proceed through the filter. For the example given of a tunnel with a 0.29° acceptance angle, scattered light within 0.58° will pass through the angular filter with a single reflection, and there is four times more scattered light within that hi-her angle than within the acceptance angle. Note that this concern is not important in the case of X-rays as they will in general not reflect from the walls while optical radiation will.

There are several methods commonly used to reduce optical reflections from surfaces, such as the depositing of an absorption film like carbon or multi-layer anti-reflection coatings. However, with the small angles involved in the high aspect ratio angular filters, absorption coatings are not as effective as they are at the larger angles of lower aspect ratio collimators. For a smooth surface the reflectivity at any given angle is given by the index of refraction of the material and the Fresnel Reflection formulas. In a the reflectivity R for light hitting a tunnel wall of index of refraction n at ala angle θ to the perpendicular of a tunnel wall is given by the modified form the Fresnel formula.

$$R = {[ \frac{sin ( θ - arc sin [ sin ( θ ) / n ]}{sin ( θ - arc sin [ sin ( θ ) / n )} ]}^{2}$$

Where R=1 is perfect reflectivity. Thus, for example, an angular filter coated with carbon would have a reflectivity for light landing perpendicularly on its surface of only 27%, but for the acceptance angle of 0.29° it would be an extremely reflective 99%. Multi-layer anti-reflection coatings use the creation of optical interference effects in the films to fully suppress reflections at specific wavelengths. However, these require careful control of film thickness, which is very dependent on the exact angles involved, and thus only work well at large angles. For example, the angular filter needing an acceptance angle of 0.29° requires a film thickness of less than 1 nm, or 1 to 2 atomic layers. Those thicknesses are very difficult and expensive to achieve. Furthermore the films no longer act like bulk material at those thicknesses and thus the optical interference effects do not occur.

At small diameters, high aspect ratios, and thus small acceptance angles, the required suppression can be achieved by changing the tunnel wall surface from a smooth surface to one generating almost total scattering of the light. One method according to the invention is to roughen the tunnel surface via specified chemical roughing or decorative etching. Alternative methods include physical processes such as ion milling. Such changes are done for two reasons: the first to increase the absorption of the surface itself, and the second to roughen the physical surface so that light rays that strike the surface at any angle will tend to reflect off or scatter at much larger angles. Thus for a long tunnel this results in light not reflecting down the tunnel, but being scattered at every point it hits the tunnel. This increases the number of reflections for shallow angle light from the scattering medium by typically tens to hundreds of times, allowing the light to be absorbed by the wall material even if its absorption level is modest. At very high or medium scattering levels, where the scattered light entering the tunnel is near to that of the ballistic or quasiballistic photons, this can reduce the amount of unwanted light passing through the tunnel by factors of four to tens and even hundreds. This significantly reduces the background noise and thus increases the image quality.

As the angular filters according to the inventions are used for such small diameter tunnels, it is vital that the roughing method used does not create particles or structures within the tunnel. Otherwise these obstructions will cause significant variation in the ballistic light reaching the detector. The chemical methods used do not create this problem if done with care.

An angular filter is provided, comprising a straight tunnel within a material, the tunnel having an entrance, an exit and a surface; wherein at least a portion of the surface of the tunnel is roughened; and wherein when the entrance to the tunnel receives a plurality of light rays, including ballistic light rays and scattered light rays, the ballistic light rays pass through the tunnel, and a plurality of scattered light rays strike the roughened surface and scatter within the tunnel and are absorbed by said material. All of the surface of the tunnel may be roughened to scatter shallow angled light. When each of the plurality of scattered light rays further scatters by the surface into a plurality of directions, the further scattered light rays strike the surface on multiple occasions, and are partially absorbed and again scattered with each strike.

The surface of the tunnel may be made of carbon or silicon. The tunnel may have a circular cross section. The roughened surface may be a pattern of ridges that reflect shallow angled light at a steeper angle, or saw tooth grooves. The surface may be roughened using chemical etching, plasma etching or ion milling.

An angular filter including a plurality of parallel tunnels within a material is provided, each of the tunnels having an entrance, an exit and a surface, wherein at least a portion of the surface of each tunnel is roughened; and wherein when each of the entrances to the tunnels receives a plurality of light rays, including ballistic light rays and scattered light rays, the ballistic light rays passing through the tunnel, and a plurality of scattered light rays striking the roughened surface of the tunnel, and scattering within the tunnel and absorbed by the material.

The material may include a lower silicon plate and an upper silicon plate, the upper silicon plate having a plurality of upward grooves, the lower silicon plate having a plurality of lower grooves, the upper grooves meeting the lower grooves to form the tunnels. The upper silicon plate may have a projection and the lower silicon plate may have a female groove sized to receive the projection, to align the upper grooves with the lower grooves.

A system for creating a two dimensional image of a scattering medium is provided, including: a light source on a first side of the scattering medium; an angular filter having a plurality of parallel tunnels within a material, each of the tunnels having an entrance, an exit and a surface, wherein at least a portion of the surface of each of the tunnels is roughened; and wherein when each of the entrances to the tunnels receives a plurality of light rays, including ballistic light rays and scattered light rays, the ballistic light rays pass through the tunnel, and a plurality of scattered light rays strike the roughened surface of the tunnel, and are reflected within the tunnel and absorbed by the material; the entrances to the tunnels facing the scattering medium on an opposite side of the scattering medium; and an imaging pixel array detector facing said exits of said angular filter. The pixels of said pixel array detector may be smaller than a distance from each of the tunnels to an adjacent tunnel, and may be smaller than the area of a cross section of the tunnels. The scattering medium may be moveable along the linear array.

A method of filtering light is provided, including providing a plurality of light rays, including ballistic light rays and scattered light rays, to an entrance to a tunnel within a material, the tunnel having an exit and a surface, wherein at least a portion of the surface of said tunnel is roughened; the ballistic light rays passing through the tunnel to the exit; and a plurality of the scattered light rays, striking the roughened surface at a shallow angle, are scattered within the tunnel and absorbed by the material.

A method of roughening a surface of a tunnel, said surface made of silicon, by altering said surface to scatter light at shallow angles into a plurality of directions. Altering the surface may include hydrating the surface with water; and immersing said surface in a solution of water and ammonium hydroxide. The ratio of water to ammonium hydroxide in the solution may be approximately 5:1. The water may be hydrated water.

The surface may be immersed in the solution for approximately 10 minutes. The method of claim 27 wherein said solution further comprises hydrofluoric acid and the ratio of water to ammonium hydroxide to hydrofluoric acid in the solution is approximately 5:1:0.1.

### DESCRIPTION OF THE DRAWINGS

For a fuller understanding of the features, advantages and objects of the invention, reference should be made to the following detailed description and the accompanying drawings.

FIG. 1 is a partial representation of one embodiment of the behavior of light in a homogenous scattering medium.

FIG. 2 is a partial representation of one embodiment of the behavior of light when detecting an object within a homogenous scattering medium.

FIG. 3 is a partial representation of one embodiment of a single tunnel angular filter.

FIG. 4 is a partial representation of an embodiment of a system for imaging within a scattering medium using a light source, a single tunnel angular filter, and a detector using transillumination methods.

FIG. 5 is a partial representation of one preferred embodiment of a micromachined high aspect ratio linear array angular filter.

FIG. 6 is a partial representation of one embodiment of a micromachined high aspect ratio angular filter linear array with circular cross sections.

FIG. 7 is a partial representation of an embodiment of the system for a linear high aspect ratio angular filter to scan and step position the filter relative to a two dimensional imaging array to create a two dimensional image of the internal structure in the scattering medium.

FIG. 8 is a partial representation of an embodiment of an angular filter tunnel with a smooth surface showing the transmittance of scattered light that just exceeds the acceptance angle.

FIG. 9 is a partial representation of an embodiment of an angular filter tunnel with a roughened surface showing the transmittance of scattered light that just exceeds the acceptance angle.

### DETAILED DESCRIPTION OF THE INVENTION

The invention will now be described with reference to the drawings in which the reference numbers designate similar features.

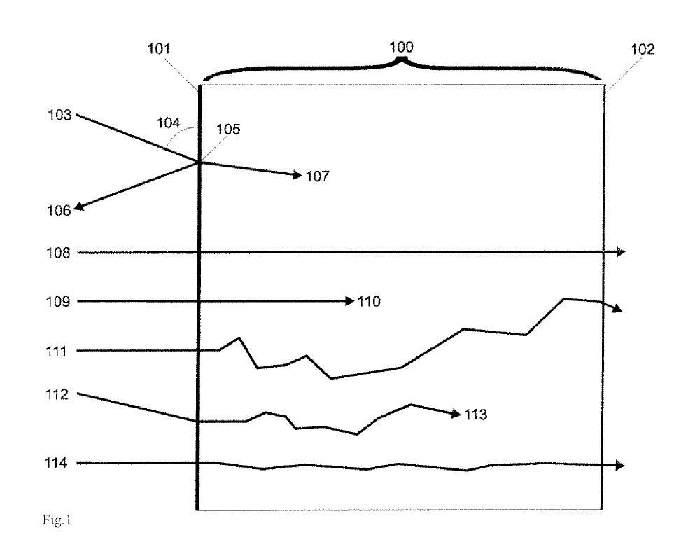

FIG. 1 is a partial representation of an embodiment of the behavior of light in a homogenous scattering medium, such as tissue. In a typical embodiment the scattering medium 100, will have a front surface 101 upon which light is impinging, and a back surface 102 from which light may exit. Examples of scattering mediums include tissue, such as human tissue, or concrete or other structural materials. Light, such as light ray 103, landing on the front surface 101 will undergo one of several behaviors. The exact behavior of the light is dependent on the direction of the light, the wavelength of the light, the index of refraction and the scattering characteristics of the medium 100. Part of the light rays, as in the case of light ray 103, that meets front surface 101 at angle 104, will meet medium 100 front surface 101 at point 105 and be reflected or scattered from front surface 101 as is light ray 106. The reflection direction and fraction of intensity of light ray 106 reflected will depend on the index of refraction of medium 100. Part of light ray 103 will also be refracted, forming a new light ray 107 in a different direction within medium 100. Light ray 108 represents an embodiment of a ballistic photon whose rays arrive perpendicular to front surface 101, and which are not reflected at surface 101 or scattered by the body of medium 100, and thus exit the medium back surface 102 in the same direction entered. For highly scattering media these ballistic rays will be a very small fraction of the light entering medium 100. Some light rays, for example light ray 109, will not exit the back surface 102 as they will be absorbed at some position 110, within medium 100. Highly scattered light rays such as light ray 11I also enter the surface perpendicular to surface 101 but are scattered by medium 100 and exit back surface 102 displaced in location and traveling in a different direction. For highly scattering media these scattered rays form the vast majority of the light. Some light rays, for example light ray 112, will be refracted, scattered and finally absorbed within medium 100, for example, at position 113. In most scattering medium the absorption rate of light is very small. Light ray 114 is an embodiment of quasi-ballistic or snake light which enters perpendicular to the medium front surface 101 and is scattered, but deviates only a small distances and angles from the ballistic path, and exits the medium back surface 102 with only a very small displacement from the entrance position. For highly scattering media this quasiballistic light is much less than the scattered light, but typically much more intense than the ballistic light.

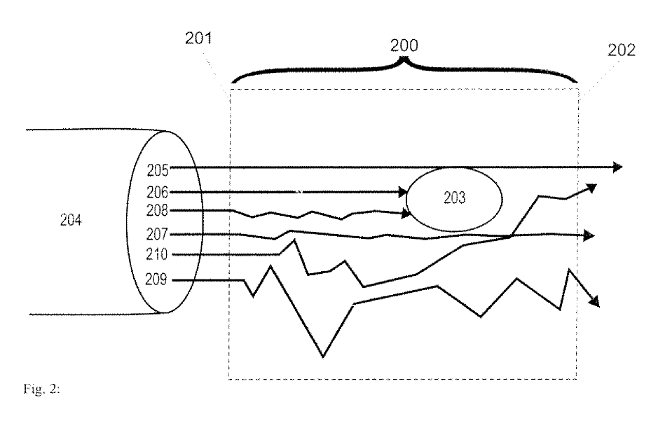

FIG. 2 is a partial representation of one embodiment of the behavior of light when detecting an object within a homogenous scattering medium. In this embodiment scattering medium 200, with front surface 201 and back surface 202, has an object or structure 203 located within medium 200. Examples of object 203 include a tumor, or an irregularity within a structure. A light source, 204, such as a laser, illuminates the front surface 201 with a number of light rays. Light ray 205 represents a ballistic light which travels through medium 200 unscattered, and is unaffected by object 203, exiting back surface 202 unaffected by the medium. Light ray 206 represents a ballistic light which travels through medium 200 unscattered, and but is blocked or absorbed by object 203, and does not exit back surface 202. Light ray 207 is a quasi-ballistic or snake light ray which is scattered by medium 200 but deviates only slightly from the ballistic path, and is unaffected by the object, existing back surface 202 with only a small position deviation. Light ray 208 is an embodiment of a quasi-ballistic or snake light ray which is scattered by medium 200 but deviates only slightly from the ballistic path, but is blocked by object 203 and does not exit surface 202. Light ray 210 is an embodiment of highly scattered light, which enters the medium at any location, and exits the back surface 202 in a nearly random position and direction. Light ray 209 is an embodiment of highly scattered light, which starts at any location, but exits the back surface 202 at a location where ballistic photons would be blocked by object 203. Since the highly scattered light rays 209 and 210 are typically millions of times more frequent than the ballistic light rays 205 and 206, or the quasiballistic light rays 207 and 208, this highly scattered light such as light ray 210 contributes the obscuring noise that should be removed in order for object 203 to be detected or imaged within medium 200. This illustrates that if scatted light could be removed and the ballistic or quasiballistic light could be observed then the object within the medium could be detected. FIG. 2 thus illustrates an embodiment of the problem of detection of an object in a highly scattering medium.

FIG. 3 is a partial representation of an embodiment of a single tunnel angular filter. The angular filter is typically made of an optically absorbing material which absorbs at least 50% of the light landing perpendicular to its surface. Two such suitable materials are carbon, or silicon, but any solid light absorbing material may be used. Highly reflective material, with reflectivity above 90%, such as aluminum or silver, would be a poor choice and transparent material, such as glass, would also be a poor choice, although the transparent material could be used if coated with an absorbent layer. In a typical embodiment of the angular filter a tunnel or tunnel of length 301 (referred to as L herein) and diameter 302 (referred to as d herein) is created through the selected material. This tunnel may be created directly through the depth of the material by many processes known in the art such as machine drilling, chemical etching, or thermal or optical processes. Alternatively the tunnel may be created by casting or molding processes, or the tunnel or may be created from the assembly of two or more parts, each of which forms the surface of the tunnel. The tunnel is preferentially hollow but may be filled with material that is not optically absorbing, provided that material does not cause light to reflect easily from the tunnel surface. Ideally the tunnel should be circular in cross section to make the detection symmetric in all directions. For such a tunnel, the aspect ratio of the tunnel is:

Aspect ratio=d/L

In an embodiment of the invention, when the angular filter is placed against or near an illumined scattering medium 303, light rays 304 leaving the medium will do so at a wide range of angles. Most of the light rays 304 will be at angles that are absorbed by the surface 300 of the tunnel. However light rays 305 and 306 represent embodiments of light rays at a sufficiently small angle to not intersect the walls of the tunnel 310 and pass through the annular filter. The limiting angle for such rays is given by the acceptance angle 307 which for geometric optics is given by:

Acceptance angle=arc tan(d/L)

Light ray 308 is an embodiment of a ballistic photon from the scattering medium which is traveling parallel to the walls of tunnel 310, and thus passes through the angular filter without being affected by it. If tunnel 310 is not circular in cross section the acceptance angle will change for different cross sections of the tunnel. Practitioners of the art will recognize that with large enough optical wavelengths there will be a diffraction affect that limits how small the diameter 302 of tunnel 310 is so that the angular filter to operate. Typically that size limit is about five times the wavelength of the light entering the tunnel.

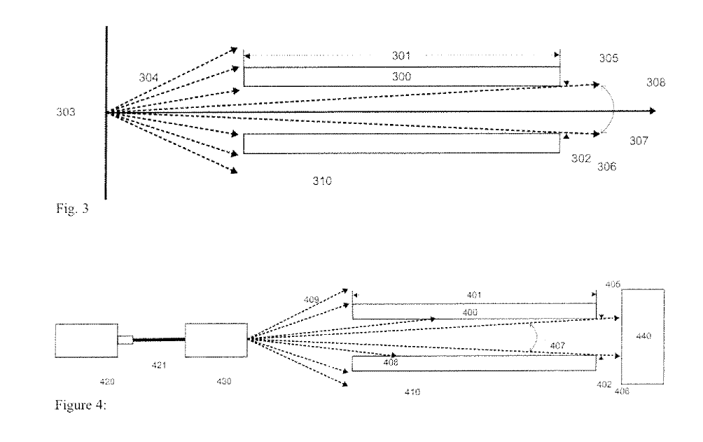

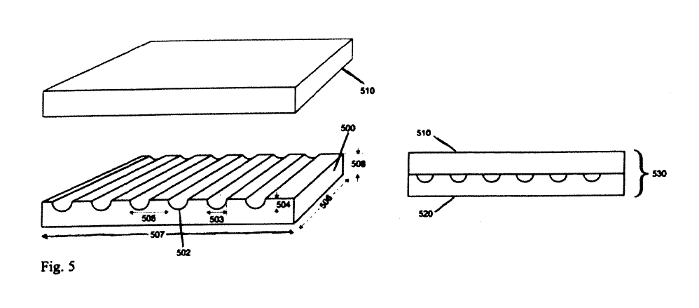

FIG. 4 is a partial representation of one particular embodiment for imaging within a scattering medium using a light source, a single tunnel angular filter, and a detector to use a transillumination method. A collimated light source 420, creates a beam of light 421 to illuminate a scattering medium 430. The collimated light source may be a laser and the additional optics needed to create collimated light beam 421. Alternatively light source 420 may be any light source where optics are applied to create the collimation. Angular filter 400, with length 401, diameter 402, and acceptance angle 407, is positioned on the opposite side of scattering medium 430 from light source 420. Angular filter 400 is aligned so that if the scattering medium 430 is removed, light 421, which would be ballistic light, passes directly through angular filter 400. In the case where the light is perfectly collimated, and thus forms a parallel beam, the surface 410 of the collimator are parallel to that beam of light. However, more commonly, the light source has a small angle of divergence. In such a case it is preferable that the light source has a divergence angle, when no scattering medium is present, of less than the angular filter acceptance angle. An optical detector or imaging array 440 is then positioned on the opposite side of the collimator from the light source. In an embodiment of the invention, this detector will be a CCD or CMOS imaging array consisting of a linear or two dimensional array of pixels. Each pixel of this array is the same size or smaller than the spacing between the tunnels of an annular filter containing more then one tunnel. Using pixels smaller than this spacing increases the resolution of the system, however ideally the number of pixels in a given tunnel is an integer multiple of that spacing, otherwise imaging artifacts are created.

FIG. 5 is a partial representation of an embodiment of a micromachined high aspect ratio linear array angular filter. The high aspect ratio angular filter 530 is created from a thin wafer of silicon using photolithographic methods and surface micromachining understood by practitioners of the art. The silicon wafer substrate is patterned and etched using acid solutions to create semicircular parallel grooves 502 of width 503, etched to a depth of 504 into the silicon, and spaced at separations of 505 in large areas of the wafer. The pattern created makes angular filter sections with these grooves that are of length 506 and cover the surface area of width 507. In a particular embodiment the pattern consists of semicircular grooves 502 with 51 μm width (503) etched to a 26 μm depth (504) with 102 μm separations (505). These are created for a 10 mm length (506) by 20 mm width (507) pattern sections on a large silicon wafer of thickness 0.5 mm (508). The wafer is then cut to create angular filter sections of 20×10 mm area. A flat smooth silicon plate 510 of the same 20×10 mm area is then placed on etched section 520. The assembled angular filter 530, as seen in this end view showing the semi-circular cross section micromachined tunnels has a 200:1 aspect ratio and 0.29° acceptance angle for its width. However note that this semicircular cross section results in the acceptance angle varying depending on whether the curved portion of the surface is struck or the flat portion of the surface. Practitioners of the art will recognize that this combination of long length with the small tunnel entry size suggest that silicon surface micromachining, best generates the structure. Silicon is also fairly light absorbent with a 40% reflectivity for visible light. Practitioners will recognize that silicon is only one possible material to use as a substrate as micromachining is often applied to many substrates of material. While in this embodiment, the tunnels are parallel along their length, less desirable structures wherein the cross sections vary along the tunnel's length are possible.

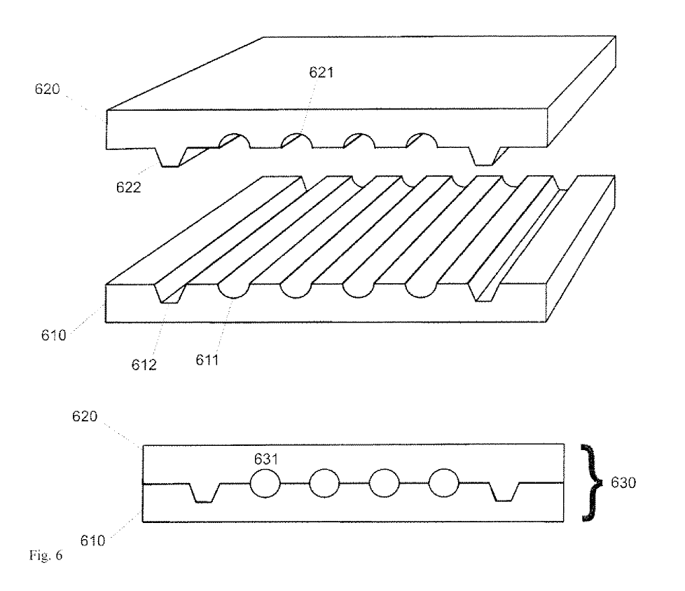

FIG. 6 is a partial representation of an embodiment of a micromachined high aspect ratio angular filter linear array with circular cross sections. In this embodiment micromachined plates, as described above with reference to FIG. 5, are used to create angular filter 630, with circular cross section tunnels 631. In this end view of angular filter 630 a bottom micromachined plate 610 with downward grooves 611 is covered with a top plate 620 with upward grooves 621, such that the downward and upward grooves align to form tunnels. In a particular embodiment these grooves are the same size as discussed with reference to FIG. 5, 51 μm width (502) tunnels are etched to a 26 μm depth (504) on 102 μm separations (505), for a 10 mm length (506) by 20 mm width (507) pattern. Practitioners of the art will recognize that if the tunnels are that size alignment of the grooves and the parallel pattern will be difficult, requiring careful positioning. Therefore the embodiment preferably includes alignment structures to the top and bottom plates to make assembly of the device easier and faster. In the particular embodiment shown female grooves 612 are micromachined into the bottom plate 610 with projections 622 created in the top plate 620. This projection 622 and groove 612 combination aligns the downward grooves 611 with the upward grooves 612. Practitioners of the art will recognize that there are many other ways of aligning the plates, such as creating grooves on both plates into which a circular wire or fiber is inserted to force the alignment. Alternatively adding simple optical alignment structures to the top and bottom allows physically micropositioning the plates so that they are in alignment.

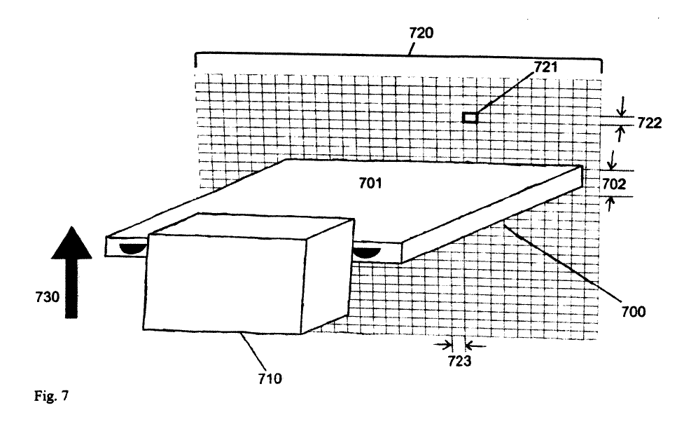

FIG. 7 is a partial representation of one embodiment of the arrangement for a linear high aspect ratio angular filter to scan and step position the filter relative to a two dimensional imaging array to create a two dimensional image of the internal structure in the scattering medium. The high aspect ratio angular filter 700, illustrated is the micromachined filter as described above with reference to FIG. 5. In front of this angular filter is placed the scattering medium 710, and to the rear of the angular filter is the imaging array 720, using the particular embodiment of the transilllumination arrangement described above in reference to FIG. 4. The pixels 721 of the imaging array 720 are aligned in rows 722 and columns 723. Each pixel of this array must be of the same size or smaller than the separation 505 of the tunnels, and is preferably smaller than the tunnel size 502. Pixels smaller than the tunnel size increase the resolution of the system. Ideally the number of pixels in a given tunnel are an integer multiple of the separation width 505. In a possible embodiment, tunnels of 51 μm in diameter and square pixels of 5.1 μm×5.1 μm are employed in an imaging array. Angular filter 700 is aligned so that the top of the flat plate 701 of the angular filter 700 is horizontally aligned with the row of the pixels 722. Angular filter 700 is also aligned perpendicular to the plane of the imaging array surface. To create a two dimensional image typically the scattering medium is moved in the vertical direction 730 in fixed distance intervals 702 that are an integer multiple of the pixels rows 721, and are close to the size of the separation 505 of the angular filter tunnels. In each interval all image is collected, and the images assembled to create the two dimensional image of the scanned scattering medium. Alternatively it is possible to move the angular filter and imager, though it is more difficult to maintain system alignment in that case.

FIG. 8 is a partial representation of an embodiment of an angular filter tunnel with a smooth surface showing the transmittance of scatter light that just exceeds the acceptance angle. In this embodiment the high aspect ratio angular filter 800 with a circular cross section and smooth tunnel wall 801, has an imaging pixel array detector 820 measuring light leaving the tunnel. As with any angular filter, ballistic light 802 passes directly through the array and is detected at position 803 at the imaging array 820. An embodiment of the scattered light 804 enters the tunnel at the shallow angle θ 805, close to that of the acceptance angle, and strikes the smooth tunnel wail 801, at point 806. At such shallow angles the scattered light is reflected off the smooth tunnel wall at 806 at the same shallow angle 805 and proceeds through the angular filter tunnel without another reflection to be detected at the imaging array 820 at point 807. This illustrates how at shallow angles where the reflectivity is high due to Fresnal reflection, the angular filter can allow significant amounts of scattered light to reach the detector. This significantly degrades the ability of the angular filter to remove scattered light, and thus increases the scattered light background noise.

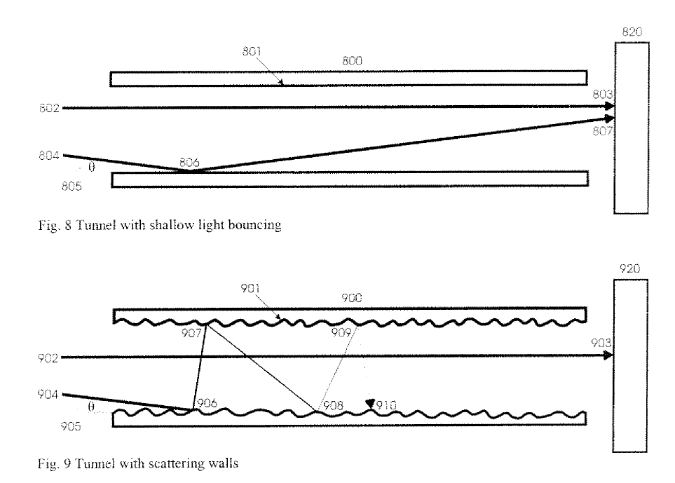

FIG. 9 is a partial representation of an embodiment of an angular filter tunnel with a roughened surface according to the invention showing the absorption of scattered light that just exceeds the acceptance angle. In this embodiment the high aspect ratio angular filter 900 with a circular cross section is used. In this particular embodiment of the invention the tunnel wall 901 has been treated to roughen its surface to make it scatter the light. As described above with reference to FIG. 8 an imaging pixel array detector 920 is present for measuring light leaving the tunnel. As with any angular filter ballistic light 902 passes directly through the array and is detected at position 903 at the imaging array 920. An embodiment of the scattered light 904 enters the tunnel at the shallow angle θ 905, slightly larger than that of the acceptance angle, and strikes the roughened tunnel wall 901, at point 906. Unlike as described above with reference to FIG. 8, in this embodiment the roughened tunnel surface scatters the light rather than passing it through the tunnel, so that it now strikes the tunnel surface at position 907. In addition the roughing effectively means the reflectivity R of the tunnel surface becomes that of the surface material at a steep angle because the true angle of the light to the roughened surface at the point of impact is no longer the shallow angle 905 of the tunnel, but a local angle at the tunnel surface at the particular location. Hence unlike as described above with reference to FIG. 8, the shallow angle light ray 904 is partially absorbed at the first reflection. The resulting reduced light intensity is shown in FIG. 9 by the decreased line thickness for the reflected light from point 906 to point 907. At point 907 the roughened surface again scatters the light to a new angle so it now intersects the tunnel at point 908, and again is shown with the resulting further reduced light intensity. At point 908 the light is again scattered to another angle, hitting the tunnel wall at point 909, and is again partially absorbed. At point 909 the beam is again scattered and absorbed by the wall at point 910. At point 910 the beam is effectively fully absorbed by the wall. Practitioners of the art will recognize that if R is the average reflectivity of the tunnel roughened wall, then after n reflections the light intensity I will be reduced from its original value I₀ by the equation

I=IₒRⁿ

In the particular embodiment where the tunnel is silicon and the average reflectivity is R=0.4 (or 40%) then after five interactions with the wall, the light in ray 904 would be reduced to about 1% of its original value. Hence the roughened tunnel surface significantly increases the ability of the angular filter to remove scattered light over the smooth walls shown in FIG. 8, and thus significantly decreases the scattered light background noise. Practitioners of the art will recognize that in practice any tunnel with smooth walls will deviate somewhat from a perfectly smooth condition. Hence there will be a small part of the light that behaves as described with reference to FIG. 9.

Practitioners of the art will recognize that in addition to the transillumination conditions described above with reference to FIG. 4, annular filters are used in other illumination conditions where the light source is not aligned with the tunnel. In those cases there are in reality no ballistic photons, and the purpose is to collect scattered light from a very limited set of angles for analysis in other ways. In these cases removing all shallow angle light greater than the acceptance angle is vital. Hence, the tunnel roughening adds significantly to the operation of those angular filters.

There are many methods to achieve the tunnel roughing described here. For example, one particular method involves use of an etching solution which attacks the material of the tunnel surfaces in an uneven manner. Many etches are isotropic, operating the same in all directions, and create a smooth removal of the material. But some etches operate unevenly, creating a patterned or convoluted surface. In the simple case etching will create ripples or patterns in the tunnel surface with many higher angle surfaces that reflect the light at much steeper angles. The ideal surface is fractal like, highly random with many very small cavities created within the surface into which light will enter, be scattered, within the cavity, and be absorbed by multiple scattering. With the high aspect collimators needing tunnel diameters in the tens of microns, these surface deviations must be in the order of a few microns. However they must not be so small that they do not affect the light, thus must be in general typically larger than one half of the wavelength of the light. The process must also not create particulates or particles that obstruct the tunnel or change the tunnel cross section from one tunnel to another in the angular filter.

Practitioners of the art will recognize that there are many etching solutions of acids or alkaline, often liquids, which will create these conditions for a given material used ill the surface of the angular filter. In the ease of angular filters made from silicon micromachining the preferred embodiment is roughening the tunnel walls using a wet chemical process very similar to what practitioners of the art call a RCA-1 clean commonly used in microfabrication, except without the use of hydrogen peroxide (H₂O₂). The preferred recipe uses a 5:1 ratio solution of water (H₂O) to ammonium hydroxide (NH₄OH), heated to a temperature of 80° C. or greater. In the case of micromachining to maintain minimize particulates that water should be filtered to the level known as Deionized Water (DI). The silicon micromachined angular filters then hydrate in DI water before being immersed in the H₂O and NH₄OH solution for 10 minutes. Also, the addition of hydrofluoric acid (HF) to the solution in the ratio of 5:1:0.1 (H₂O:NH₄OH:HF) was observed to improve the uniformity of the roughening. While these ratios may be preferable, ratios near these values will work, but often with less effectiveness.

There are many other methods of creating this roughing, including plasma or reactive ion etching of the surface. Alternatively a physical reaction such as surface sputtering or ion milling may be used to create random patterns on the surface. Note that the surface does not need to be random but can contain patterns such as ridges that reflect the light at much steeper angles or a series of saw tooth grooves. In addition simple mechanical processes as machine milling, scoring the walls with a hard tool, hitting the surface with small particles (sand blasting), mechanically rubbing rough material materials (in a simple case sand paper) or solutions containing particles that wear away the surface could be employed, or wearing the surface away with electrical current methods. However in general those mechanical methods create too large a surface roughness for the small diameters of high aspect ratio angular filters, and tend to produce particles that partially block the tunnel. Application of films which create the desired highly convoluted, scattering surfaces is also possible, but again the challenge is to prevent such film creating particles and having sufficiently small surface structures. Microfabrication methods such as sputter coating, or simple film evaporation can create such small films. Angular filters with such rough surfaces can also be created by making a master with a roughened surface, creating a mold from that master, and using molding methods to reproduce the rough surface. The important point there is to make certain that all the mold material is removed from the tunnel wall so that it remains rough.

As will be apparent to those skilled in the art in the light of the foregoing disclosure, many alterations and modifications are possible in the practice of this invention without departing from the spirit or scope thereof. Accordingly, the scope of the invention is to be construed in accordance with the substance defined by the following claims.

1. An angular filter, comprising: a straight tunnel within a material, said tunnel having an entrance, an exit and a surface; wherein at least a portion of said surface of said tunnel is roughened; and wherein when said entrance to said tunnel receives a plurality of light rays, said light rays including ballistic light rays and scattered light rays, said ballistic light rays pass through said tunnel to said exit, and a plurality of scattered light rays, strike said roughened surface, and scatter within said tunnel and are absorbed by said material.

2. The angular filter of claim 1 wherein all of said surface of said tunnel is roughened to scatter shallow angled light.

3. The angular filter of claim 2, wherein the surface of said tunnel is made of carbon.

4. The angular filter of claim 2 wherein the surface of said tunnel is made of silicon.

5. The angular filter of claim 4 wherein said tunnel has a circular cross section.

6. The angular filter of claim 2, wherein each of said plurality of scattered light rays further scatters by said tunnel surface into a plurality of directions, such that said further scattered light rays strike said tunnel surface on multiple occasions, and said further scattered light rays are partially absorbed and again scattered with each said strike.

7. The angular filter of claim 6 wherein said roughened surface is a pattern of ridges that reflect shallow angled light at a steeper angle.

8. The angular filter of claim 6 wherein said roughened surface is a pattern of saw tooth grooves.

9. The angular filter of claim 2 wherein said surface is roughened using chemical etching.

10. The angular filter of claim 2 wherein said surface is roughened using plasma etching.

11. The angular filter of claim 2 wherein said surface is roughened using ion milling.

12. An angular filter comprising a plurality of parallel tunnels within a material, each of said tunnels having an entrance, an exit and a surface, wherein at least a portion of said surface of each of said tunnels is roughened; and wherein when each of said entrances to said tunnels receives a plurality of light rays, said light rays including ballistic light rays and scattered light rays, said ballistic light rays pass through said tunnel to said exit, and a plurality of scattered light rays, strike said roughened surface of said tunnel, and scatter within said tunnel and are absorbed by said material.

13. The angular filter of claim 12 wherein all of said surface of each of said tunnels is roughened to scatter shallow angled light.

14. The angular filter of claim 13 wherein the surface of said tunnels is made of silicon.

15. The angular filter of claim 14 wherein each of said tunnels has a circular cross section.

16. The angular filter of claim 15 wherein said material comprising a lower silicon plate and an upper silicon plate, said upper silicon plate having a plurality of upward grooves, said lower silicon plate having a plurality of lower grooves, said upper grooves meeting said lower grooves to form said tunnels.

17. The angular filter of claim 16 wherein said upper silicon plate has a projection and said lower silicon plate has a female groove sized to receive said projection, to align said upper grooves with said lower grooves.

18. A system for creating a two dimensional image of a scattering medium comprising:
a. a light source on a first side of said scattering medium;
b. an angular filter comprising a plurality of parallel tunnels within a material, each of said tunnels having an entrance, an exit and a surface, wherein at least a portion of said surface of each of said tunnels is roughened; and wherein when each of said entrances to said tunnels receives a plurality of light rays, said light rays including ballistic light rays and scattered light rays, said ballistic light rays pass through said tunnel to said exit, and a plurality of scattered light rays, strike said roughened surface of said tunnel, and are reflected within said tunnel and absorbed by said material; said entrances to said tunnels facing said scattering medium on an opposite side of said scattering medium; and
c. an imaging pixel array detector facing said exits of said angular filter.

19. The system of claim 18 wherein the pixels of said pixel array detector are smaller than a distance from each of said tunnels to an adjacent tunnel.

20. The system of claim 19 wherein said pixels are smaller than the area of a cross section of said tunnels.

21. The system of claim 20 wherein said scattering medium is moveable along said linear array.

22. A method of filtering light comprising:
(a) providing a plurality of light rays, including ballistic light rays and scattered light rays, to an entrance to a tunnel within a material, said tunnel having an exit and a surface, wherein at least a portion of said surface of said tunnel is roughened;
(b) said ballistic light rays passing through said tunnel to said exit; and
(c) a plurality of said scattered light rays, striking said roughened surface at a shallow angle, are scattered within said tunnel and absorbed by said material.

23. A method of roughening a surface of a tunnel, said surface made of silicon, by altering said surface to scatter light at shallow angles into a plurality of directions.

24. The method of claim 23 wherein altering said surface comprises:
a) hydrating said surface in water; and
b) immersing said surface in a solution of water and ammonium hydroxide.

25. The method of claim 24, wherein the ratio of water to ammonium hydroxide in said solution is approximately 5:1.

26. The method of claim 25 wherein said water is deionized water.

27. The method of claim 26 wherein said surface is immersed in said solution for approximately 10 minutes.

28. The method of claim 27 wherein said solution further comprises hydrofluoric acid.

29. The method of claim 28 wherein the ratio of water to ammonium hydroxide to hydrofluoric acid in said solution is approximately 5:1:0.1.
